(19) United States
(12) Patent Application Publication
(10) Pub. No.: US 2020/0268247 A1
(43) Pub. Date: Aug. 27, 2020

(54) BIOLOGICAL INFORMATION PROCESSING APPARATUS

(71) Applicant: SONY CORPORATION, TOKYO (JP)

(72) Inventors: MASANORI IWASAKI, KANAGAWA (JP); NAOTO YAMAGUCHI, TOKYO (JP); TSUKASA YOSHIMURA, TOKYO (JP); MASAKAZU YAJIMA, KANAGAWA (JP); FUMIKO SHIGA, TOKYO (JP); KEN HAYAKAWA, KANAGAWA (JP)

(21) Appl. No.: 16/753,693

(22) PCT Filed: Aug. 6, 2018
(86) PCT No.: PCT/JP2018/029410

§ 371 Date: Apr. 3, 2020

(30) Foreign Application Priority Data
Oct. 13, 2017 (JP) 2017-199361

(51) Int. Cl. A61B 3/113; A61B 3/10; A61B 3/16; G16H 50/30

(52) U.S. Cl. CPC A61B 3/113; A61B 3/101; G16H 20/60; G16H 50/30; A61B 3/16

(57) ABSTRACT

A biological information processing apparatus according to an embodiment of the present disclosure includes a generating section and an output section. The generating section generates an analysis result according to a request of a user on a basis of first biological information about a tear, an eyeball, or a line of sight obtained using a contact lens. The output section outputs the analysis result generated by the generating section to an external apparatus.

### TECHNICAL FIELD

The present disclosure relates to a biological information processing apparatus.

### BACKGROUND ART

In recent years, a method has been developed for acquiring biological information using a contact lens.

### CITATION LIST

### Patent Literature

PTL 1: Japanese Unexamined Patent Application Publication No. 2017-125914

### SUMMARY OF THE INVENTION

Incidentally, there is a demand for more effective use of biological information acquired using a contact lens. It is therefore desirable to provide a biological information processing apparatus that makes it possible to more effectively use the biological information acquired using the contact lens.

A biological information processing apparatus according to an embodiment of the present disclosure includes a generating section and an output section. The generating section generates an analysis result according to a request of a user on the basis of first biological information about a tear, an eyeball, a line of sight, or opening/closing of an eyelid obtained using a contact lens. The output section outputs the analysis result generated by the generating section to an external apparatus.

In the biological information processing apparatus according to an embodiment of the present disclosure, the analysis result according to the request of the user is generated on the basis of the first biological information about the tear, the eyeball, the line of sight, or the opening/closing of the eyelid obtained using the contact lens. This makes it possible to provide the user with the analysis result effectively usable by the user.

According to the biological information processing apparatus of an embodiment of the present disclosure, the analysis result according to the request of the user is generated on the basis of the first biological information about the tear, the eyeball, the line of sight, or the opening/closing of the eyelid obtained using the contact lens, thus making it possible to more effectively use the biological information acquired using the contact lens. It is to be noted that effects of the present disclosure are not necessarily limited to the effects described above, and may be any of the effects described in the present specification.

### BRIEF DESCRIPTION OF THE DRAWINGS

FIG. 1 illustrates an example of a schematic configuration of a biological information management system according to an embodiment of the present disclosure.

FIG. 2 illustrates an example of functional blocks of a contact lens illustrated in FIG. 1.

FIG. 3 illustrates an example of functional blocks of a cradle illustrated in FIG. 1.

FIG. 4 illustrates an example of functional blocks of a terminal apparatus illustrated in FIG. 1.

FIG. 5 illustrates an example of functional blocks of a data server apparatus illustrated in FIG. 1.

FIG. 6 illustrates an example of functional blocks of a web server apparatus illustrated in FIG. 1.

FIG. 7 illustrates an example of a procedure of transmitting and receiving detection data in the biological information management system illustrated in FIG. 1.

FIG. 8 illustrates an example of a procedure of transmitting and receiving request data and analysis data in the biological information management system illustrated in FIG. 1.

FIG. 9 illustrates a modification example of the schematic configuration of the biological information management system illustrated in FIG. 1.

FIG. 10 illustrates a modification example of the schematic configuration of the biological information management system illustrated in FIG. 1.

FIG. 11 illustrates an example of functional blocks of a wearable apparatus illustrated in FIG. 10.

FIG. 12 illustrates an example of a procedure of transmitting and receiving detection data in the biological information management system illustrated in FIG. 10.

FIG. 13 illustrates a modification example of the schematic configuration of the biological information management system illustrated in FIG. 1.

### MODES FOR CARRYING OUT THE INVENTION

In the following, modes for carrying out the present disclosure are described in detail with reference to the drawings. It is to be noted that the description is given in the following order.
1. Embodiment

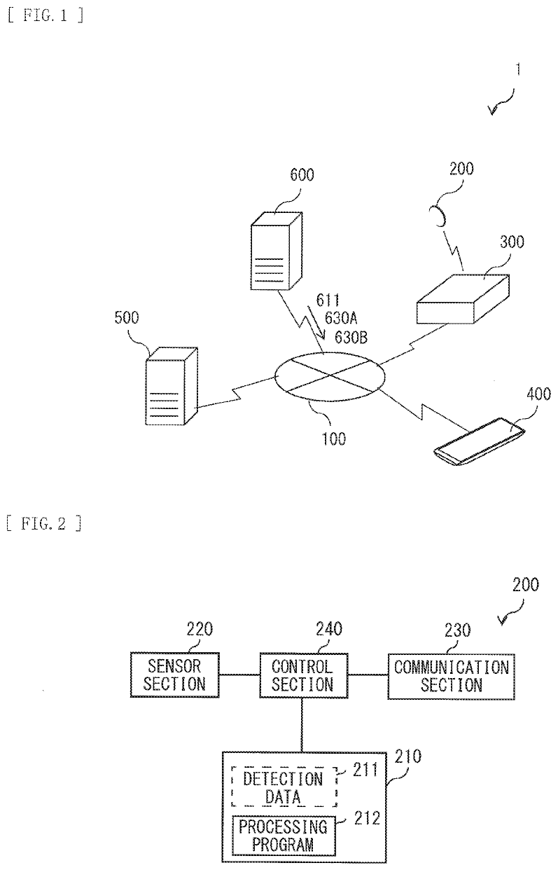

Example of transmitting data to a data server apparatus via a cradle (FIG. 1 to FIG. 8)
2. Modification Examples

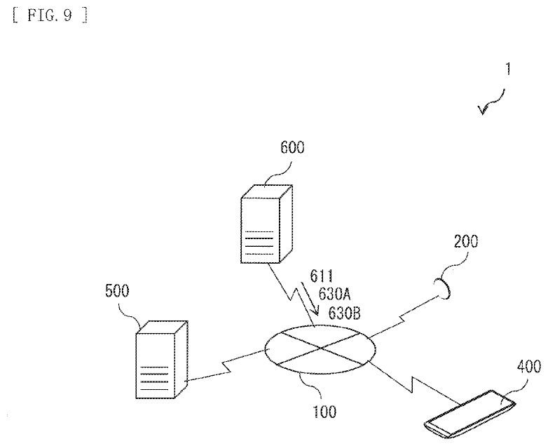

Modification Example A . . . Example of transmitting data directly to a data server apparatus (FIG. 9)

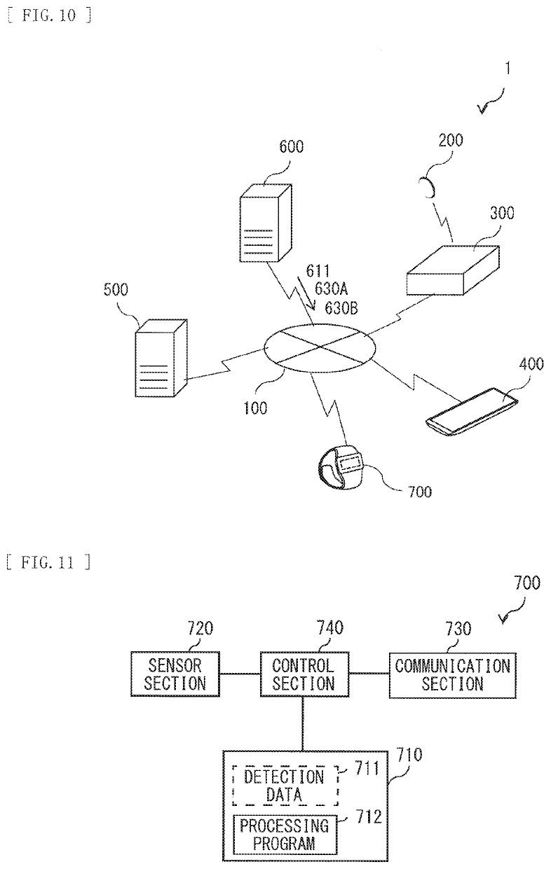

Modification Example B . . . Example of acquiring biological information from a contact lens and a wearable apparatus (FIG. 10 to FIG. 13)

### 1. EMBODIMENT

### [Configuration]

Description is given of a biological information management system 1 according to an embodiment of the present disclosure. FIG. 1 illustrates a schematic configuration example of the biological information management system 1. The biological information management system 1 is a system that performs analysis according to a user request on the basis of biological information of the user and outputs a result thereof to a mobile terminal (external apparatus) possessed by the user or the like.

The biological information management system 1 includes a contact lens 200, a cradle 300, a terminal apparatus 400, a data server apparatus 500, and a web server apparatus 600. The cradle 300 corresponds to a specific example of a “biological information processing apparatus” of the present disclosure. Moreover, the web server apparatus 600 corresponds to a specific example of a “biological information processing apparatus” of the present disclosure.

The cradle 300, the terminal apparatus 400, the data server apparatus 500, and the web server apparatus 600 are coupled to one another via a network 100. The network 100 is a communication network that performs communication using a communication protocol (TCP/IP) used as a standard in the Internet, for example. The network 100 may be, for example, a secure communication network that performs communication using a communication protocol unique to the network. The network 100 is, for example, the Internet, an intranet, or a local area network. The network 100 may be, for example, a wired LAN (Local Area Network), or may be a wireless LAN such as Wi-Fi or a mobile phone line.

Meanwhile, the contact lens 200 performs, for example, short-range wireless communication with the cradle 300. The contact lens 200 performs short-range wireless communication with the cradle 300, for example, in a state of being fixed to a predetermined location in a casing of the cradle 300. Communication between the contact lens 200 and the cradle 300 is performed on the basis of, for example, ISO/IEC14443 (international standard specification of proximity RFID), ISO/IEC18092 (international specification of wireless communication referred to as NFC), ISO/IEC15693 (international standard specification of RFID), Bluetooth (Bluetooth (registered trademark)), or the like.

### (Contact Lens 200)

FIG. 2 illustrates an example of functional blocks of the contact lens 200. The contact lens 200 includes, for example, a storage section 210, a sensor section 220, a communication section 230, and a control section 240.

The communication section 230 performs short-range wireless communication with the cradle 300. The storage section 210 includes, for example, a non-volatile memory, such as an EEPROM (Electrically Erasable Programmable Read-Only Memory), a flash memory, a resistive random-access memory, or the like. The storage section 210 stores therein a program (e.g., a processing program 212) to be executed by the control section 240, and the like. The processing program 212 is a program for acquiring detection data 211 that is biological information from the sensor section 220 and for outputting the acquired detection data 211 to the cradle 300 via the communication section 230. The detection data 211 is data obtained using the contact lens 200, and is specifically biological information detected by the sensor section 220. The control section 240 includes a processor, and executes the processing program 212 stored in the storage section 210, for example. A function of the control section 240 is achieved by, for example, the control section 240 executing the processing program 212.

The sensor section 220 obtains biological information of a user wearing the contact lens 200. The sensor section 220 is a device that detects a specific component (e.g., salt, oxygen, fat, or glucose level) contained in a tear, for example. In this case, the data (detection data 211) obtained by being detected by the sensor section 220 is information (first biological information) about the component of a tear. It is to be noted that the sensor section 220 may be, for example, a device that detects a line of sight, a device that detects a state of a blood vessel in an eyeball, a device that detects a pulse of the blood vessel in the eyeball, or a device that detects an intraocular pressure. In a case where the sensor section 220 is the device that detects the line of sight, the detection data 211 is the biological information (first biological information) about the line of sight. In a case where the sensor section 220 is the device that detects the state of the blood vessel in the eyeball, the detection data 211 is the information (first biological information) about the blood vessel in the eyeball. In a case where the sensor section 220 is the device that detects the intraocular pressure, the detection data 211 is the information (first biological information) about the intraocular pressure. The sensor section 220 may be, for example, a device that detects opening/closing of an eyelid. In a case where the sensor section 220 is the device that detects the opening/closing of the eyelid, the detection data 211 is the information (first biological information) about the opening/closing of the eyelid.

### (Cradle 300)

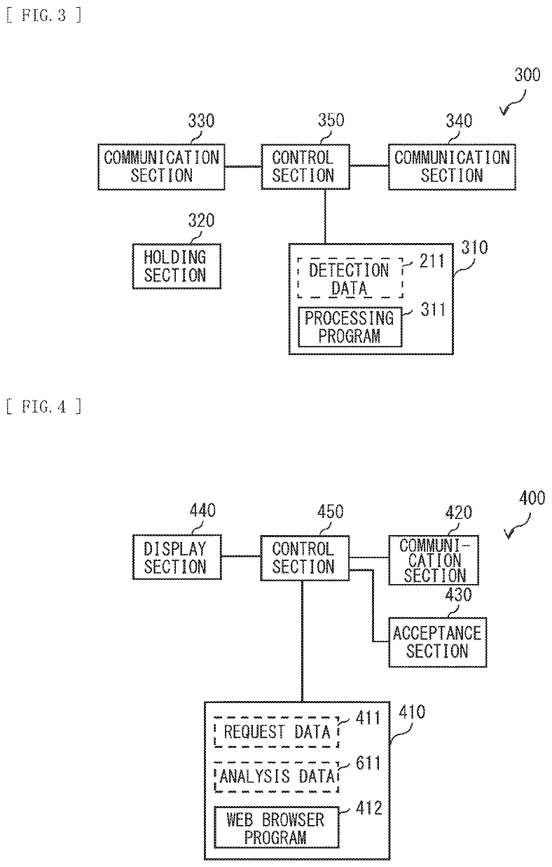

FIG. 3 illustrates an example of functional blocks of the cradle 300. The cradle 300 includes, for example, a storage section 310, a holding section 320, communication sections 330 and 340, and a control section 350. The holding section 320 corresponds to a specific example of a “holding section” of the present disclosure. The control section 350 and the communication section 330 correspond to a specific example of an “acquiring section” of the present disclosure. The control section 350 and the communication section 340 correspond to a specific example of an “output section” of the present disclosure.

The holding section 320 is provided inside the casing of the cradle 300, and holds the contact lens 200. The communication section 330 performs short-range wireless communication with the contact lens 200 held by the holding section 320. Specifically, the communication section 330 acquires the detection data 211 that is biological information from the storage section 210 of the contact lens 200 held by the holding section 320. The communication section 340 performs communication with the web server apparatus 600 via the network 100. Specifically, the communication section 340 outputs the detection data 211 obtained by the communication section 330 to the web server apparatus 600 via the network 100.

The storage section 310 includes, for example, a non-volatile memory, such as an EEPROM, a flash memory, a resistive random-access memory, or the like. The storage section 310 stores therein a program (e.g., a processing program 311) to be executed by the control section 350, and the like. The processing program 311 is a program for acquiring the detection data 211 from the contact lens 200 via the communication section 330 and for outputting the acquired detection data 211 to the web server apparatus 600 via the communication section 340 and the network 100. The control section 350 includes a processor, and executes the processing program 311 stored in the storage section 310, for example. A function of the control section 350 is achieved by, for example, the control section 350 executing the processing program 311.

### (Terminal Apparatus 400)

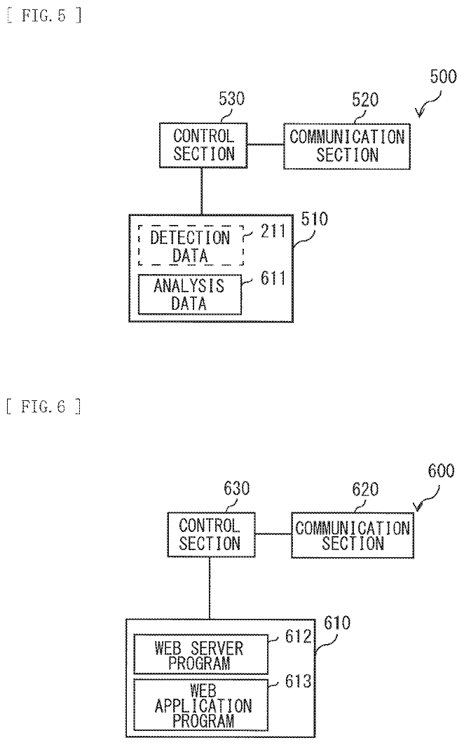

FIG. 4 illustrates an example of functional blocks of the terminal apparatus 400. The terminal apparatus 400 is a terminal apparatus used for allowing the user to input a request required for analysis of biological information and for displaying an analysis result according to the user request. The terminal apparatus 400 includes an interface for accepting a request from the user and displaying the analysis result according to the user request. The terminal apparatus 400 includes, for example, a storage section 410, a communication section 420, an acceptance section 430, a display section 440, and a control section 450.

The acceptance section 430 includes a keyboard, a touch panel, or the like. The display section 440 performs display on the basis of an image signal inputted from the control section 450. In a case where the display section 440 has a function of the touch panel, the function of the touch panel may assume a function of the acceptance section 430. The storage section 410 stores therein a program (e.g., a web browser program 412 and an operating system) to be executed by the control section 450, and the like. The storage section 410 stores therein request data 411 and analysis data 611.

The request data 411 is data about a request accepted from the user. The request from the user includes, for example, a request for management of performance or condition (hereinbelow, referred to as “request A”) based on the biological information (first biological information) of the user. It is to be noted that the request from the user may include a request for a proposal of a training menu, a food menu, or a nutrition menu (hereinbelow, referred to as “request B”) based on the biological information (first biological information) of the user. Moreover, the request from the user may include a request for a notice of presence/absence of a symptom of a disease (hereinbelow, referred to as “request C”) based on the first biological information of the user.

The analysis data 611 is data about analysis result according to the request data 411. In a case where the request data 411 is the “request A,” the analysis data 611 is data about the performance or the condition of the user. In a case where the request data 411 is the “request B,” the analysis data 611 is data about the proposal of the training menu, the food menu, or the nutrition menu for the user. In a case where the request data 411 is the “request C,” the analysis data 611 is data about the symptom of the disease of the user.

The control section 450 includes a processor, and executes the web browser program 412, the operating system, and the like stored in the storage section 410. The communication section 420 communicates with another apparatus (such as the web server apparatus 600 and the cradle 300) inside the biological information management system 1 via the network 100. A user interface (display section 440) in the terminal apparatus 400 is achieved by, for example, processing an application (e.g., html data) acquired from the web server apparatus 600 with the web browser program 412.

### (Data Server Apparatus 500)

FIG. 5 illustrates an example of functional blocks of the data server apparatus 500. The data server apparatus 500 is, for example, an apparatus for storing therein the detection data 211 obtained using the contact lens 200 and the analysis data 611 obtained by data processing in the web server apparatus 600.

The data server apparatus 500 includes, for example, a storage section 510, a communication section 520, and a control section 530. The control section 530 stores in the storage section 510 information inputted from the outside via the communication section 520 and outputs information read out from the storage section 510 to the outside via the communication section 520. The communication section 520 communicates with another apparatus via the network 100. The storage section 510 includes, for example, a non-volatile memory, such as an EEPROM, a flash memory, a resistive random-access memory, or the like. The storage section 510 stores therein, for example, the detection data 211 and the analysis data 611.

### (Web Server Apparatus 600)

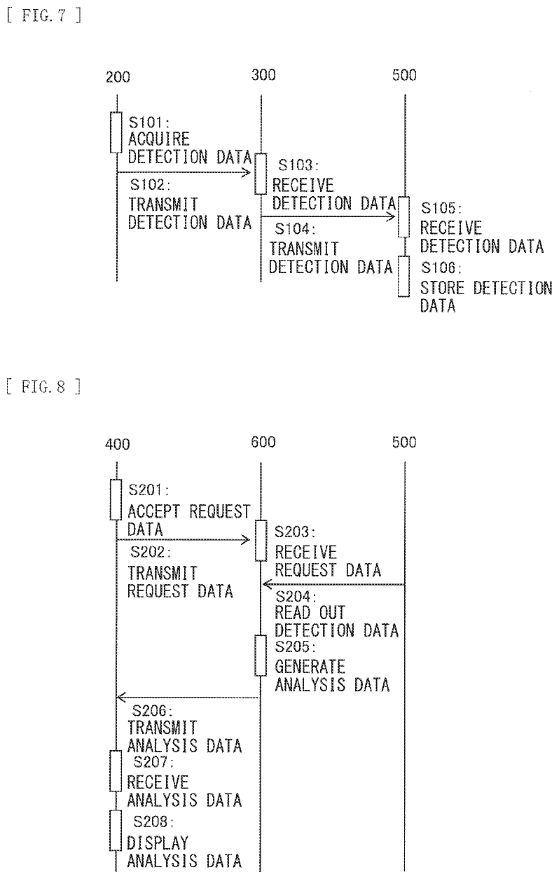

FIG. 6 illustrates an example of functional blocks of the web server apparatus 600. The web server apparatus 600 provides a user interface in the terminal apparatus 400. The web server apparatus 600 further generates the analysis result (analysis data 611) according to the request data 411 accepted from the user on the basis of the detection data 211 obtained using the contact lens 200. The web server apparatus 600 includes, for example, a storage section 610, a communication section 620, and a control section 630. The control section 630 corresponds to a specific example of a “generating section” of the present disclosure. The control section 630 and the communication section 620 correspond to a specific example of the “output section” of the present disclosure.

The communication section 620 communicates with another apparatus (e.g., the data server apparatus 500, the terminal apparatus 400, and the like) inside the biological information management system 1 via the network 100. The storage section 610 includes, for example, a non-volatile memory, such as an EEPROM, a flash memory, a resistive random-access memory, or the like. The storage section 610 stores therein a program (e.g., a web server program 612 and a web application program 613) to be executed by the control section 630. The web application program 613 is an application program used via the network 100. The web application program 613 is a program that generates the analysis result (analysis data 611) according to the request data 411 accepted from the user on the basis of the detection data 211 obtained using the contact lens 200. The web application program 613 is operated by the web server program 612 and the web browser program 412 cooperating with each other.

The control section 630 includes a processor, and executes the web server program 612, the web application program 613, and the like stored in the storage section 610, for example. A function of the control section 630 is achieved by, for example, the control section 630 executing the web server program 612 and the web application program 613. Specifically, the control section 630 may generate the analysis result (analysis data 611) according to the request data 411 accepted from the user on the basis of the detection data 211 obtained using the contact lens 200.

The control section 630 may generate, when the detection data 211 satisfies a predetermined condition, a control signal 630A for notifying thereof, and output the generated control signal 630A to the terminal apparatus 400 via the communication section 520. The “predetermined condition” refers to, for example, a condition that requires attention drawing for the performance or the condition of the user. The control signal 630A is, for example, text data that draws attention.

### [Operation]

### (Storage of Detection Data)

Next, a procedure of storing the detection data 211 in the biological information management system 1 is described.

FIG. 7 illustrates an example of a procedure of transmitting and receiving the detection data 211 in the biological information management system 1. The user first puts the contact lens 200 on his or her eyeball. Thereafter, the sensor section 220 acquires the biological information (detection data 211) of the user in the contact lens 200 (Step S101). In a case where the sensor section 220 is the device that detects the specific component (e.g., salt, oxygen, fat, or glucose level) contained in a tear, the detection data 211 is the biological information about the tear. In a case where the sensor section 220 is the device that detects the line of sight, the detection data 211 is the biological information about the line of sight. In a case where the sensor section 220 is the device that detects the state of the blood vessel in the eyeball, the detection data 211 is the biological information about the eyeball. In a case where the sensor section 220 is the device that detects the intraocular pressure, the detection data 211 is the biological information about the eyeball. In a case where the sensor section 220 is the device that detects the opening/closing of an eyelid, the detection data 211 is the biological information about the opening/closing of the eyelid.

The sensor section 220 transmits the acquired biological information (detection data 211) to the cradle 300 (Step S102). When receiving the detection data 211 from the sensor section 220 (Step S103), the cradle 300 transmits the received detection data 211 to the data server apparatus 500 (Step S104). When receiving the detection data 211 from the cradle 300 (Step S105), the data server apparatus 500 stores the received detection data 211 in the storage section 510 (Step S106).

### (Analysis of Detection Data)

Next, a procedure of analyzing the detection data 211 in the biological information management system 1 is described.

FIG. 8 illustrates an example of a procedure of analyzing the detection data 211 in the biological information management system 1. The user first requests the terminal apparatus 400 to display the user interface for inputting a request. The terminal apparatus 400 then displays the user interface for inputting the request on the display section 440 by processing an application acquired from the web server apparatus 600 with the web browser program 412. The user inputs the request data 411 using the displayed user interface and the acceptance section 430. When accepting the request data 411 inputted by the user (Step S201), the terminal apparatus 400 transmits the accepted request data 411 to the web server apparatus 600 (Step S202).

When receiving the request data 411 from the terminal apparatus 400 (Step S203), the web server apparatus 600 reads out the detection data 211 corresponding to contents of the received request data 411 from the data server apparatus 500 (Step S204). The web server apparatus 600 generates the analysis result (analysis data 611) according to the request data 411 on the basis of the read-out detection data 211 (Step S205). The web server apparatus 600 transmits the generated analysis data 611 to the terminal apparatus 400 (Step S206). When receiving the analysis data 611 from the web server apparatus 600 (Step S207), the terminal apparatus 400 displays the received analysis data 611 on the display section 440 (Step S208).

### [Effects]

Next, effects of the biological information management system 1 according to the present embodiment are described.

In the present embodiment, the analysis data 611 according to the request data 411 is generated on the basis of the detection data 211 about a tear, an eyeball, a line of sight, or opening/closing of an eyelid obtained using the contact lens 200. This makes it possible to provide the user with the analysis result (analysis data 611) effectively usable by the user. As a result, the user is allowed to manage the performance or the condition of the user himself or herself on the basis of the analysis data 611, for example. Accordingly, it is possible to more effectively use the biological information (detection data 211) acquired using the contact lens 200.

Moreover, in the present embodiment, the detection data 211 includes information about a component of the tear, the blood vessel in the eyeball, the intraocular pressure, the line of sight, or the opening/closing of the eyelid. This allows the analysis data 611 to be obtained on the basis of the component of the tear, the blood vessel in the eyeball, the intraocular pressure, the line of sight, or the opening/closing of the eyelid, thus enabling the user to manage the performance or the condition of the user himself or herself on the basis of the analysis data 611, for example. Accordingly, it is possible to more effectively use the biological information (detection data 211) acquired using the contact lens 200.

Moreover, in the present embodiment, in a case where the request data 411 is the “request A,” it is possible to obtain the analysis result (analysis data 611) allowing for effective management of the performance or the condition of the user. As a result, the user is allowed to manage the performance or the condition of the user himself or herself on the basis of the analysis data 611, for example. Accordingly, it is possible to more effectively use the biological information (detection data 211) acquired using the contact lens 200.

Moreover, in the present embodiment, in a case where the request data 411 is the “request B,” it is possible to obtain the analysis result (analysis data 611) allowing for effective management of the training, the food, or the nutrition of the user. As a result, the user is allowed to manage the training, the food, or the nutrition of the user on the basis of the analysis data 611, for example. Accordingly, it is possible to more effectively use the biological information (detection data 211) acquired using the contact lens 200.

Moreover, in the present embodiment, in a case where the request data 411 is the “request C,” it is possible to obtain the analysis result (analysis data 611) allowing for effective management of the symptom of the disease of the user. As a result, the user is allowed to manage the symptom of the disease of the user on the basis of the analysis data 611, for example. Accordingly, it is possible to more effectively use the biological information (detection data 211) acquired using the contact lens 200.

Moreover, in the present embodiment, when the detection data 211 satisfies a predetermined condition, the control section 530 generates the control signal 630A for notifying thereof, and in a case where the generated control signal 630A is outputted to the terminal apparatus 400 via the communication section 520, it is possible to promptly notify the user that the detection data 211 satisfies the predetermined condition. As a result, the user is allowed to effectively manage a change (accident) in the performance or the condition of the user on the basis of the control signal 630A, for example. Accordingly, it is possible to more effectively use the biological information (detection data 211) acquired using the contact lens 200.

Moreover, in the present embodiment, the detection data 211 is acquired from the storage section 210 of the contact lens 200 held by the holding section 320 in the cradle 300, and the acquired detection data 211 is outputted to the web server apparatus 600. This makes it possible to reduce signal strength and power consumption required from the contact lens 200 as compared with a case of transmitting the detection data 211 to the data server apparatus 500 without using the cradle 300. Accordingly, it is possible to manage the performance or the condition of the user at lower cost.

Moreover, in the present embodiment, the detection data 211 is acquired from the storage section 210 of the contact lens 200 through the short-range wireless communication in the cradle 300. This makes it possible to reduce the signal strength and the power consumption required from the contact lens 200 as compared with the case of transmitting the detection data 211 to the data server apparatus 500 without using the cradle 300. Accordingly, it is possible to manage the performance or the condition of the user at lower cost.

Moreover, in the present embodiment, the detection data 211 acquired by the control section 350 in the cradle 300 is outputted to the web server apparatus 600 via the communication network 100. This makes it possible to transmit the detection data 211 to the data server apparatus 500 more reliably as compared with the case of transmitting the detection data 211 to the data server apparatus 500 without using the cradle 300. Accordingly, it is possible to manage the performance or the condition of the user more reliably.

### 2. MODIFICATION EXAMPLES

### Modification Example A

In the above-described embodiment, the contact lens 200 may not have the storage section 210, the sensor section 220, the communication section 230, and the control section 240, but have a structure for storing tears therein. In this case, the cradle 300 may have, instead of the communication section 330, for example, a light source that emits light to the tears stored in the contact lens 200 held by the holding section 320 and a light receiving section that detects light having passed through the tears from among the light emitted to the tears from the light source. Furthermore, in the cradle 300 having the light source and the light receiving section, the control section 350 may analyze concentration of the specific component (e.g., salt, oxygen, fat, or glucose level) contained in the tear, for example, on the basis of a detection signal outputted from the light receiving section as a result of detection of light in the light receiving section. In a case where the cradle 300 has such a configuration, it is possible to effectively manage the performance or the condition of the user while minimizing an influence on a human body, as compared with a case where the contact lens 200 has a communication function.

### Modification Example B

In the above-described embodiment, the contact lens 200 may be configured to communicate directly with the data server apparatus 500 via the network 100 without the cradle 300 as illustrated in FIG. 9, for example. Even in such a case, it is possible to provide the user with the analysis result (analysis data 611) effectively usable by the user, thus making it possible to more effectively use the biological information (detection data 211) acquired using the contact lens 200.

### Modification Example C

In the above-described embodiment, the control section 630 may be configured to further generate an advice 630B according to the request data 411 on the basis of the detection data 211 and output the generated advice 630B to the terminal apparatus 400 via the communication section 620. In this case, for example, it is possible to obtain an advice useful for improving the performance or the condition of the user without the need for the user himself or herself to consider the analysis data 611, for example. Accordingly, it is possible to effectively manage the performance or the condition of the user, for example.

### Modification Example D

In the above-described embodiment, the biological information management system 1 may further include a wearable apparatus 700 wearable by the user as illustrated in FIG. 10, for example. The wearable apparatus 700 is, for example, a watch, a head gear, a bracelet, an anklet, a necklace, an earring, or a shoe incorporating an electronic component as illustrated in FIG. 11 described below.

### (Wearable Apparatus 700)

FIG. 11 illustrates an example of functional blocks of the wearable apparatus 700. The wearable apparatus 700 is configured to communicate with the data server apparatus 500 via the network 100. The wearable apparatus 700 includes, for example, a storage section 710, a sensor section 720, a communication section 730, and a control section 740.

The communication section 730 communicates with the web server apparatus 600 via the network 100. Specifically, the communication section 730 outputs detection data 711 generated by the sensor section 720 to the web server apparatus 600 via the network 100. The storage section 710 includes, for example, a non-volatile memory, such as an EEPROM, a flash memory, a resistive random-access memory, or the like. The storage section 710 stores therein a program (e.g., a processing program 712) to be executed by the control section 740. The processing program 712 is a program for acquiring the detection data 711 that is biological information from the sensor section 720 and for outputting the acquired detection data 711 to the data server apparatus 500 via the communication section 730 and the network 100. The detection data 711 is data obtained using the wearable apparatus 700 (sensor section 720), and is specifically biological information detected by the sensor section 720. The control section 740 includes a processor, and executes the processing program 712 stored in the storage section 710, for example. A function of the control section 740 is achieved by, for example, the control section 740 executing the processing program 712.

The sensor section 720 acquires the biological information of the user wearing the wearable apparatus 700. The sensor section 720 is a device that detects, for example, a brain wave, a pulse, body temperature, sweat, or a posture of the user. In this case, the data (detection data 711) obtained by being detected by the sensor section 720 is information (second biological information) about the brain wave, the pulse, the body temperature, the sweat, or the posture.

### [Operation]

### (Storage of Detection Data)

Next, a procedure of storing the detection data in the biological information management system 1 according to the present modification example is described.

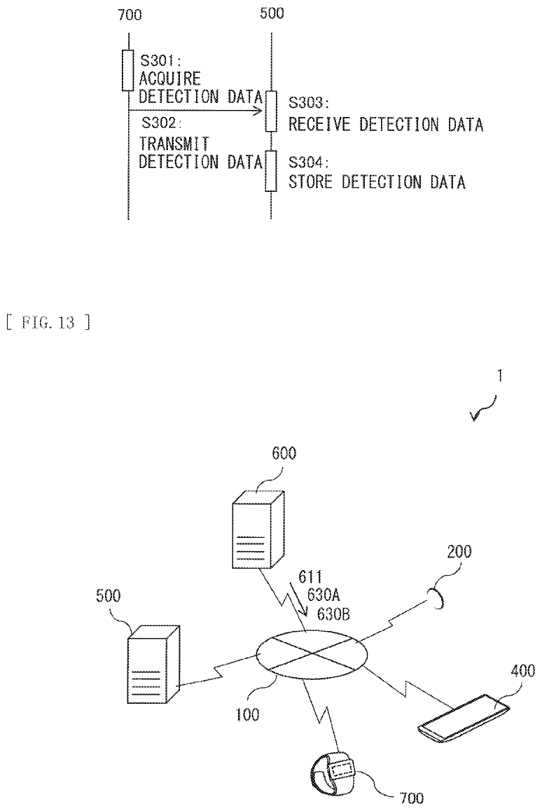

FIG. 12 illustrates an example of a procedure of transmitting and receiving the detection data 211 in the biological information management system 1 according to the present modification example. The user first puts the wearable apparatus 700 on his or her eyeball. Thereafter, the sensor section 720 acquires the biological information (detection data 711) of the user in the wearable apparatus 700 (Step S301). In a case where the sensor section 720 is the device that detects the brain wave, the detection data 711 is the biological information about the brain wave. In a case where the sensor section 720 is the device that detects the pulse, the detection data 711 is the biological information about the pulse. In a case where the sensor section 720 is the device that detects the body temperature, the detection data 711 is the biological information about the body temperature. In a case where the sensor section 720 is the device that detects the sweat, the detection data 711 is the biological information about the sweat. In a case where the sensor section 720 is the device that detects the posture, the detection data 711 is the biological information about the posture.

The sensor section 720 transmits the acquired biological information (detection data 711) to the data server apparatus 500 via the network 100 (Step S302). When receiving the detection data 711 from the wearable apparatus 700 (Step S303), the data server apparatus 500 stores the received detection data 711 in the storage section 510 (Step S304).

### (Analysis of Detection Data)

Next, a procedure of analyzing the detection data in the biological information management system 1 according to the present modification example is described.

The user first requests the terminal apparatus 400 to display the user interface for inputting a request. The terminal apparatus 400 then displays the user interface for inputting the request on the display section 440 by processing the application acquired from the web server apparatus 600 with the web browser program 412. The user inputs the request data 411 using the displayed user interface and the acceptance section 430. When accepting the request data 411 inputted by the user (Step S201, FIG. 8), the terminal apparatus 400 transmits the accepted request data 411 to the web server apparatus 600 (Step S202, FIG. 8).

When receiving the request data 411 from the terminal apparatus 400 (Step S203, FIG. 8), the web server apparatus 600 reads out the detection data 211 and 711 corresponding to the contents of the received request data 411 from the data server apparatus 500 (Step S204, FIG. 8). The web server apparatus 600 generates the analysis result (analysis data 611) according to the request data 411 on the basis of the read-out detection data 211 and 711 (Step S205, FIG. 8). The web server apparatus 600 transmits the generated analysis data 611 to the terminal apparatus 400 (Step S206, FIG. 8). When receiving the analysis data 611 from the web server apparatus 600 (Step S207, FIG. 8), the terminal apparatus 400 displays the received analysis data 611 on the display section 440 (Step S208, FIG. 8).

### [Effects]

Next, effects of the biological information management system 1 according to the present modification example are described.

In the present modification example, the analysis data 611 according to the request data 411 is generated on the basis of the detection data 211 obtained using the contact lens 200 and the detection data 711 obtained using the wearable apparatus 700 worn by the user. This makes it possible to provide the user with the analysis result (analysis data 611) effectively usable by the user. As a result, the user is allowed to manage the performance or the condition of the user himself or herself on the basis of the analysis data 611, for example. Accordingly, it is possible to more effectively use the biological information (detection data 211) acquired using the contact lens 200 and the information (detection data 711) acquired using the wearable apparatus 700.

In the present modification example, the detection data 711 includes the information about the brain wave, the pulse, the body temperature, the sweat, or the posture. This allows the analysis data 611 to be obtained on the basis of the brain wave, the pulse, the body temperature, the sweat, or the posture, thus enabling the user to manage the performance or the condition of the user himself or herself on the basis of the analysis data 611, for example. Accordingly, it is possible to more effectively use the biological information (detection data 211) acquired using the contact lens 200 and the information (detection data 711) acquired using the wearable apparatus 700.

In the present modification example, the control section 530 may be configured to further generate the advice 630B according to the request data 411 on the basis of the detection data 211 and output the generated advice 630B to the terminal apparatus 400 via the communication section 520. In this case, for example, it is possible to obtain an advice useful for improving the performance or the condition of the user without the need for the user himself or herself to consider the analysis data 611, for example. Accordingly, it is possible to effectively manage the performance or the condition of the user, for example.

In the present modification example, the contact lens 200 may be configured to communicate directly with the data server apparatus 500 via the network 100 without the cradle 300 as illustrated in FIG. 13, for example. Even in such a case, it is possible to provide the user with the analysis result (analysis data 611) effectively usable by the user, thus making it possible to more effectively use the biological information (detection data 211) acquired using the contact lens 200 and the information (detection data 711) acquired using the wearable apparatus 700.

Although the present disclosure has been described hereinabove with reference to the embodiment and modification examples thereof, the present disclosure is not limited to the above-described embodiment, etc., and may be modified in a variety of ways.

In the above-described embodiment and the modification examples thereof, the sensor section 720 acquires the biological information of the user wearing the wearable apparatus 700. However, in the above-described embodiment and the modification examples thereof, the sensor section 720 may acquire impact, speed, moving distance, or oxygen concentration in the atmosphere. In this case, the web server apparatus 600 (control section 630) estimates an amount of exercise and an action (e.g., exercise contents, work contents, a state such as being bedridden or hospitalized) of the user on the basis of at least one of the impact, the speed, the moving distance, or the oxygen concentration in the atmosphere stored as the detection data 711 in the data server apparatus 500. The sensor section 720 may further acquire a voice of the user. In this case, the web server apparatus 600 (control section 630) determines the situation of the user on the basis of at least one of the impact, the speed, the moving distance, and the oxygen concentration in the atmosphere stored as the detection data 711 in the data server apparatus 500 as well as the voice, and then estimates the amount of exercise and the action (e.g., exercise contents, work contents, the state such as being bedridden or hospitalized) of the user.

The web server apparatus 600 (control section 630) transmits information about the estimated amount of exercise and the estimated action of the user, as the analysis data 611, to the terminal apparatus 400. As a result, the user is allowed to perform, for example, management of actions of the user in a day and consideration on lacking exercise and rehabilitation.

It is to be noted that the effects described in the present specification are merely exemplary. The effects of the present disclosure are not limited to those described in the present specification. The present disclosure may have any other effects than those described in the present specification.

In addition, for example, the present technology may have the following configurations.
(1)

A biological information processing apparatus including:

a generating section that generates an analysis result according to a request of a user on a basis of first biological information about a tear, an eyeball, a line of sight, or opening/closing of an eyelid obtained using a contact lens; and

an output section that outputs the analysis result generated by the generating section to an external apparatus.
(2)

The biological information processing apparatus according to (1), in which the first biological information includes information about a component of the tear, a blood vessel in the eyeball, an intraocular pressure, the line of sight, or the opening/closing of the eyelid.
(3)

The biological information processing apparatus according to (1) or (2), in which the request includes management of performance or a condition of the user based on the first biological information.
(4)

The biological information processing apparatus according to (1) or (2), in which the request includes a proposal of a training menu, a food menu, or a nutrition menu of the user based on the first biological information.
(5)

The 1 biological information processing apparatus according to (1) or (2), in which the request includes a notice of presence/absence of a symptom of a disease of the user based on the first biological information.
(6)

The biological information processing apparatus according to (5), in which

the generating section generates, when the first biological information satisfies a predetermined condition, a control signal for notifying thereof, and

the output section outputs the control signal generated by the generating section to the external apparatus.
(7)

The biological information processing apparatus according to any one of (1) to (6), in which

the generating section further generates an advice according to the request on the basis of the first biological information, and

the output section outputs the advice generated by the generating section to the external apparatus.
(8)

The biological information processing apparatus according to any one of (1) to (6), in which the generating section generates the analysis result on a basis of the first biological information and second biological information obtained using a wearable apparatus worn by the user.
(9)

The biological information processing apparatus according to (8), in which the second biological information includes information about a brain wave, a pulse, body temperature, sweat, or a posture.
(10)

The biological information processing apparatus according to (8) or (9), in which

the generating section further generates an advice according to the request on the basis of the first biological information and the second biological information, and

the output section outputs the advice generated by the generating section to the external apparatus.

This application claims the benefit of Japanese Priority Patent Application JP2017-199361 filed with the Japanese Patent Office on Oct. 13, 2017, the entire contents of which are incorporated herein by reference.

It should be understood by those skilled in the art that various modifications, combinations, sub-combinations and alterations may occur depending on design requirements and other factors insofar as they are within the scope of the appended claims or the equivalents thereof.

1. A biological information processing apparatus comprising:
a generating section that generates an analysis result according to a request of a user on a basis of first biological information about a tear, an eyeball, a line of sight, or opening/closing of an eyelid obtained using a contact lens; and
an output section that outputs the analysis result generated by the generating section to an external apparatus.

2. The biological information processing apparatus according to claim 1, wherein the first biological information includes information about a component of the tear, a blood vessel in the eyeball, an intraocular pressure, the line of sight, or the opening/closing of the eyelid.

3. The biological information processing apparatus according to claim 1, wherein the request comprises management of performance or a condition of the user based on the first biological information.

4. The biological information processing apparatus according to claim 1, wherein the request comprises a proposal of a training menu, a food menu, or a nutrition menu of the user based on the first biological information.

5. The biological information processing apparatus according to claim 1, wherein the request comprises a notice of presence/absence of a symptom of a disease of the user based on the first biological information.

6. The biological information processing apparatus according to claim 5, wherein
the generating section generates, when the first biological information satisfies a predetermined condition, a control signal for notifying thereof, and
the output section outputs the control signal generated by the generating section to the external apparatus.

7. The biological information processing apparatus according to claim 1, wherein
the generating section further generates an advice according to the request on the basis of the first biological information, and
the output section outputs the advice generated by the generating section to the external apparatus.

8. The biological information processing apparatus according to claim 1, wherein the generating section generates the analysis result on a basis of the first biological information and second biological information obtained using a wearable apparatus worn by the user.

9. The biological information processing apparatus according to claim 8, wherein the second biological information includes information about a brain wave, a pulse, body temperature, sweat, or a posture.

10. The biological information processing apparatus according to claim 8, wherein
the generating section further generates an advice according to the request on the basis of the first biological information and the second biological information, and
the output section outputs the advice generated by the generating section to the external apparatus.
